(19) United States
(12) Patent Application Publication
(10) Pub. No.: US 2025/0252620 A1
(43) Pub. Date: Aug. 7, 2025

(54) SYSTEM AND METHOD OF PRODUCING A TOMOGRAM

(71) Applicant: Adaptix Ltd, Begbroke (GB)

(72) Inventors: Mark Evans, Witney (GB); Martin Holden, Begbroke (GB); Conrad Dirckx, Begbroke (GB); Andy Barnes, Begbroke (GB)

(21) Appl. No.: 19/191,087

(22) Filed: Apr. 28, 2025

Related U.S. Application Data
(63) Continuation of application No. PCT/GB2023/052565, filed on Oct. 4, 2023.

(30) Foreign Application Priority Data
Oct. 28, 2022 (GB) 2215976.8

(51) Int. Cl. G06T 11/00; A61B 6/03; G06V 10/74

(52) U.S. Cl. CPC G06T 11/005; A61B 6/032; G06V 10/761; G06V 2201/07

(57) ABSTRACT

A method to identify and/or assess structural integrity of a composite material, by distributing fiducial markers within the composite material during manufacture, and subsequently creating x-ray 3D tomosynthesis images of the composite material using an array of x-ray emitters and a digital x-ray detector, the 3D tomosynthesis images being used to determine the relative location of at least some of the fiducial markers. This enables detection of counterfeit products by comparison with recorded data at manufacture and detection of an internal degradation of the material indicated by movement of the fiducial markers. Relative locations of fiducial markers in a first set of images are compared with relative locations of fiducial markers in a second set of images, to identify common fiducial markers and thereby determine an offset therebetween. Both sets of images may be used to reconstruct a combined three-dimensional density function.

### RELATED APPLICATIONS

This application claims the benefit under 35 U.S.C. § 120, and is a continuation, of co-pending International Application PCT/GB2023/052565, filed Oct. 4, 2023 and designating the US, which claims priority to GB Application 2215976.8, filed Oct. 28, 2022, such GB Applications also being claimed priority to under 35 U.S.C. § 119. These GB and International applications are incorporated by reference herein in their entireties.

### FIELD

The present invention relates generally to a system and method of producing a tomogram and finds particular, although not exclusive, utility in producing 3D tomosynthesis images of at least a portion of a composite material, particularly in the non-destructive evaluation and testing of composite materials with an aim of identifying structural failures and/or counterfeit materials.

### BACKGROUND

Composite materials are generally defined as consisting of two or more materials, combined in such a way that the composite's properties are distinct from those of the individual materials. Common examples include fibre-reinforced plastics and carbon fibre but can also include plastic-metal laminates and other laminates or matrix materials.

Non-destructive evaluation and testing of components and particularly of components containing composites is challenging. For example, delamination is a mode of failure where a material fractures into layers. A variety of materials including laminate composites can fail by delamination.

Structural Health Monitoring (SHM) may be defined as the “acquisition, validation and analysis of technical data to facilitate life-cycle management decisions.” More generally, SHM denotes a reliable system with the ability to detect and interpret adverse “changes” in a structure due to damage or normal operation.

SHM is more advantageous to some industries, such as the aerospace industry, since damage can lead to catastrophic (and expensive) failures, and the vehicles involved have regular costly inspections. Aircraft are increasingly including composite materials to take advantage of their excellent specific strength and stiffness properties, as well their ability to reduce radar cross-section and “part-count”. The disadvantage, however, is that composite materials present challenges for design, maintenance and repair over metallic parts since they tend to fail by distributed and interacting damage modes. Furthermore, damage detection in composites is much more difficult due to the anisotropy of the material, the conductivity of the fibres, the insulative properties of the matrix, and the fact that much of the damage often occurs beneath the top surface of the laminate, for instance with barely visible impact damage.

Currently successful composite non-destructive testing techniques for small laboratory specimens, such as radiographic detection (penetrant enhanced X-ray) and hydro-ultrasonics (C-scan), are impractical for large components and integrated vehicles.

Furthermore, the main limitation of current visualisation techniques is a very limited possibility to image so-called closed delamination in which delaminated layers are in contact practically with no physical gap.

Several techniques have been researched for detecting damage in composite materials focused on modal response. These methods are among the earliest and most common, principally because they are simple to implement on any size structure. Structures can be excited by ambient energy, an external shaker or embedded actuators, and embedded strain gauges, piezometers or accelerometers can be used to monitor the structural dynamic responses. Changes in normal vibrational modes can be correlated to loss of stiffness in a structure, and usually analytical models or experimentally determined response-history tables are used to predict the corresponding location of damage. The difficulty, however, comes in the interpretation of the data collected by this type of system. There are also detection limitations imposed by the resolution and range of the individual sensors chosen, and the density with which they are distributed over the structure.

Another area of interest is that of 3D printing, or additive manufacturing, where often a single material is applied, layer by layer, to build up an object. While conventional 3D printing may not be considered a composite in the traditional sense, the layered structure has similar challenges to laminates in that they have low x-ray contrast, can suffer from hidden voids and flaws.

### SUMMARY

A problem with such products is that of “ply wrinkling” with various causes including thermal history, shifting of the vacuum bag, non-uniform resin, etc. These wrinkles may render a part unfit, but such wrinkling may go undetected until late in the manufacturing process (adding significant costs to the cast-off part) or entirely undetected (leading to an unsuitable part being deployed in the field). Therefore, detection of such wrinkles and related defects during manufacture is of key interest.

Ultrasound provides limited information about structural integrity of many types of parts and can easily fail in complex assemblies. Two-dimension x-rays do not reveal flaws in structures with complex overlying and underlying layers. Existing 3D x-ray imaging (i.e. CT) can be slow, expensive, heavy and very complex to field as it requires three-phase power and a radiation shielded room. Also, CT typically use high doses of radiation which may damage some sensitive components. Conventional mechanical tests (strain gauges, magnaflux, etc.) often do not work well with additive manufacturing and can fail to reveal hidden flaws until failure occurs.

A method has been recently developed to identify and/or assess structural integrity of a composite material, by distributing fiducial markers (which attenuate x-rays to an extent greater than the rest of the material) within the composite material during manufacture, and subsequently creating x-ray 3D tomosynthesis images of the composite material using an array of x-ray emitters and a digital x-ray detector, the 3D tomosynthesis images being used to determine the relative location of at least some of the fiducial markers with respect to one another. This enables detection of counterfeit products by comparison with recorded data at manufacture, and also enables detection of an internal degradation of the material indicated by movement of the fiducial markers. However, large structures cannot be imaged in a single set of exposures and so individual parts of the structures must be assessed separately.

According to a first aspect of the present invention, there is provided a method of producing a tomogram, the method comprising the steps of: acquiring a first set of x-ray attenuation images of an object over a first region; identifying a first plurality of fiducial markers in the first set of x-ray attenuation images; acquiring a second set of x-ray attenuation images of the object over a second region; identifying a second plurality of fiducial markers in the second set of x-ray attenuation images; comparing relative locations of the first plurality of fiducial markers with relative locations of the second plurality of fiducial markers, and thereby identifying a plurality of common fiducial markers present in the first plurality of fiducial markers and the second plurality of fiducial markers; using locations of the plurality of common fiducial markers in the first and second sets of x-ray attenuation images to determine an offset between the first region and the second region; and reconstructing a combined three-dimensional density function indicative of attenuation of x-ray radiation from the first set of x-ray attenuation images and the second set of x-ray attenuation images.

In this way, a 3D tomosynthesis image of an entire structure may be formed more efficiently in real time.

A tomogram is an image produced by tomography, and in the context of the present application may be produced by tomosynthesis, digital tomosynthesis (DTS) and/or high-resolution limited-angle tomography.

Acquiring may comprise emitting x-ray radiation from an emitter and detecting it at a detector. The or each region may comprise a three-dimensional volume, for example between an emitter and a detector.

The fiducial markers may comprise a first material that attenuates x-rays to an extent greater than a second material in which the fiducial markers are embedded. The fiducial markers may comprise one or more of copper, iron, molybdenum, tungsten and gold. Other elements or compounds may be employed as they provide contrast with resinous material (e.g. epoxy or polyester resins) and fibre (e.g. carbon or glass) when imaged using x-rays. The fiducial markers may comprise ceramics material and/or metallic material (for example carbon nanotubes with metallic cores). Other ceramic/metallic materials (or other attenuating markers) may be introduced into the resinous material when the composite is being formed at a level that will not negatively impact the functional properties of the device with respect to strength and weight. It is also possible that a carbon nanotube is ‘tagged’ with an attenuating marker. This may be effected by not completing the standard carbon nanotube manufacture process thus leaving a ferrous molecule on the inside of the carbon nanotube. It is also possible to have one or more metal sheaths or metal particle “decorations” on the carbon nanotube. These may result from additional processing steps, such as the application of coatings, etc.

The fiducial markers may comprise particles having a size of approximately 1 to 40 μm. Other sizes such as in the range 50-5000 nm are contemplated.

The resinous material may comprise approximately less than 0.1% by weight of the fiducial markers.

The fiducial markers may be invisible to the naked eye from outside the material.

The ratio of fiducial markers to resinous material, by volume, may vary through the material to provide an indication of their location. For instance, the ratio may increase or decrease through the material from one side to an opposite side. For example, the ratio may increase or decrease with each layer of material (if the material has been formed in an additive manufacturing manner). Determining the ratio at any given point in the material (by means of x-ray imaging) may provide an indication of location within the material.

The quantity of fiducial markers within the resinous material may vary in a controlled manner through the material. The term “controlled manner” includes a regular increase/decrease in quantity with position, however, other changes in quantity may be included too, such as a logarithmic increase/decrease, and an increase/decrease controlled by a known algorithm. Determining the quantity at any given point in the material (by means of x-ray imaging) may provide an indication of location within the material.

Likewise, the size and/or composition of the fiducial markers within the resinous material may vary in a controlled manner through the material. Determining the size and/or composition at any given point in the material (by means of x-ray imaging) may provide an indication of location within the material.

The fiducial markers may be arranged regularly throughout the resinous material, or at defined intervals on the fibre within a composite. For instance, a regular 2D pattern may be produced in each layer to create an overall 3D pattern. This may more easily assist in determining de-lamination or ply-wrinkling of layered materials.

Alternatively, the fiducial markers may simply be identifiable elements in the acquired images, and may be naturally occurring.

Relative locations of the fiducial markers may mean, for example, the spacings between said fiducial markers. Alternatively or additionally, relative locations of the fiducial markers may mean a location relative to an emitter or detector of the x-rays.

Comparing relative locations of the first plurality of fiducial markers with relative locations of the second plurality of fiducial markers, and thereby identifying a plurality of common fiducial markers present in the first plurality of fiducial markers and the second plurality of fiducial markers, may comprise creating a first point cloud from the relative locations of the first plurality of fiducial markers, creating a second point cloud from the relative locations of the second plurality of fiducial markers, and performing point cloud registration therebetween.

Point cloud registration may comprise finding a spatial transformation (e.g. scaling, rotation and/or translation) that aligns at least a subset of the first point cloud with a subset of the second point cloud.

Determine the offset between the first region and the second region may therefore comprise extrapolating such an offset from the spatial transformation.

The offset may comprise a distance, direction, orientation and/or scaling of the second region with respect to the first region, or vice versa.

Reconstruction may comprise use of any suitable reconstruction algorithm, including filtered back projection algorithms and iterative reconstruction algorithms.

The first set of x-ray attenuation images may comprise a single first x-ray attenuation image only.

The first set of x-ray attenuation images may comprise a plurality of first x-ray attenuation images.

The method may further comprise the step of reconstructing a first three-dimensional density function indicative of attenuation of x-ray radiation from the first set of x-ray attenuation images, and the step of identifying the first plurality of fiducial markers in the first set of x-ray attenuation images may comprises identifying the first plurality of fiducial markers in the first three-dimensional density function.

The second set of x-ray attenuation images may comprise a single second x-ray attenuation image only.

The second set of x-ray attenuation images may comprise a plurality of second x-ray attenuation images.

The method may further comprise the step of reconstructing a second three-dimensional density function indicative of attenuation of x-ray radiation from the second set of x-ray attenuation images, and the step of identifying the second plurality of fiducial markers in the second set of x-ray attenuation images may comprise identifying the second plurality of fiducial markers in the second three-dimensional density function.

Reconstructing the combined three-dimensional density function may comprise determining an emitter offset and/or a detector offset between first position(s) from which the first set of x-ray attenuation images is acquired and second position(s) from which the second set of x-ray attenuation images is acquired.

The first/second position(s) may comprise a first/second emitter location and/or a first/second emitter orientation of an emitter panel from which x-rays are emitted, and/or may comprise a first/second detector location and/or a first/second detector orientation of a detector panel from which x-rays are detected. The first/second emitter/detector location/orientation may comprise a plurality of such locations/orientations corresponding to the respective locations of individual emitter/detectors in the emitter/detector panel, respectively.

The method may further comprise identifying a combined plurality of fiducial markers in the combined three-dimensional density function.

The method may further comprise allocating as a combined plurality of fiducial markers, the first plurality of fiducial markers and the second plurality of fiducial markers.

The method may further comprise the steps of: acquiring a third set of x-ray attenuation images of the object over a third region; identifying a third plurality of fiducial markers in the third set of x-ray attenuation images; comparing relative locations of the combined plurality of fiducial markers with relative locations of the third plurality of fiducial markers, and thereby identifying a further plurality of common fiducial markers present in the combined plurality of fiducial markers and the third plurality of fiducial markers; using locations of the further plurality of common fiducial markers to determine a further offset between the first and/or second region and the third region; and/or reconstructing a further combined three-dimensional density function indicative of attenuation of x-ray radiation from the first set of x-ray attenuation images, the second set of x-ray attenuation images and the third set of x-ray attenuation images.

The third set of x-ray attenuation images may comprise a single third x-ray attenuation image only.

The third set of x-ray attenuation images may comprise a plurality of third x-ray attenuation images.

The method may further comprise the step of reconstructing a third three-dimensional density function indicative of attenuation of x-ray radiation from the third set of x-ray attenuation images, and the step of identifying the third plurality of fiducial markers in the third set of x-ray attenuation images may comprise identifying the third plurality of fiducial markers in the third three-dimensional density function.

Reconstructing the combined three-dimensional density function may be achieved in the absence of the first, second and/or third three-dimensional density function.

Reconstructing the further combined three-dimensional density function may comprise determining a further emitter offset and/or a further detector offset between first and/or second position(s) from which the first and/or second sets of x-ray attenuation images are acquired and third position(s) from which the third set of x-ray attenuation images is acquired.

The third position(s) may comprise a third emitter location and/or a third emitter orientation of an emitter panel from which x-rays are emitted, and/or may comprise a third detector location and/or a third detector orientation of a detector panel from which x-rays are detected. The third emitter/detector location/orientation may comprise a plurality of such locations/orientations corresponding to the respective locations of individual emitter/detectors in the emitter/detector panel, respectively.

According to a second aspect of the present invention, there is provided a system for producing a tomogram, the system comprising: an x-ray detector panel; an x-ray emitter panel comprising an array of x-ray emitters permanently fixed with respect to each other, each x-ray emitter configured to emit a respective cone of x-rays therefrom to impinge on the x-ray detector panel; and a processor configured to operate the x-ray detector and emitter panels to carry out the method of any of the preceding.

### BRIEF DESCRIPTION OF THE ACCOMPANYING DRAWINGS

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

FIG. 1 is a perspective view of an x-ray imaging apparatus.

### DETAILED DESCRIPTION

The present invention will be described with respect to certain drawings but the invention is not limited thereto but only by the claims. The drawings described are only schematic and are non-limiting. Each drawing may not include all of the features of the invention and therefore should not necessarily be considered to be an embodiment of the invention. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that operation is capable in other sequences than described or illustrated herein. Likewise, method steps described or claimed in a particular sequence may be understood to operate in a different sequence.

Moreover, the terms top, bottom, over, under and the like in the description and the claims are used for descriptive purposes and not necessarily for describing relative positions. It is to be understood that the terms so used are interchangeable under appropriate circumstances and that operation is capable in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “an embodiment” or “an aspect” means that a particular feature, structure or characteristic described in connection with the embodiment or aspect is included in at least one embodiment or aspect of the present invention. Thus, appearances of the phrases “in one embodiment”, “in an embodiment”, or “in an aspect” in various places throughout this specification are not necessarily all referring to the same embodiment or aspect, but may refer to different embodiments or aspects. Furthermore, the particular features, structures or characteristics of any one embodiment or aspect of the invention may be combined in any suitable manner with any other particular feature, structure or characteristic of another embodiment or aspect of the invention, as would be apparent to one of ordinary skill in the art from this disclosure, in one or more embodiments or aspects.

Similarly, it should be appreciated that in the description various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Moreover, the description of any individual drawing or aspect should not necessarily be considered to be an embodiment of the invention. Rather, as the following claims reflect, inventive aspects lie in fewer than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some features included in other embodiments, combinations of features of different embodiments are meant to be within the scope of the invention, and form yet further embodiments, as will be understood by those skilled in the art. For example, in the following claims, any of the claimed embodiments can be used in any combination.

In the description provided herein, numerous specific details are set forth. However, it is understood that embodiments of the invention may be practised without these specific details. In other instances, well-known methods, structures and techniques have not been shown in detail in order not to obscure an understanding of this description.

In the discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The use of the term “at least one” may mean only one in certain circumstances. The use of the term “any” may mean “all” and/or “each” in certain circumstances.

The principles of the invention will now be described by a detailed description of at least one drawing relating to exemplary features. It is clear that other arrangements can be configured according to the knowledge of persons skilled in the art without departing from the underlying concept or technical teaching, the invention being limited only by the terms of the appended claims.

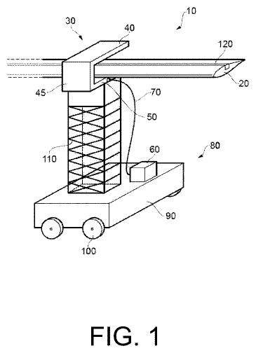

In FIG. 1, an X-ray imaging apparatus 10 is depicted comprising a C-arm 30 having an upper arm 40, a lower arm 50 and a connecting portion 45. However, it is to be appreciated that the apparatus 10 could be embodied by having independently movable emitter and detector arms, or a combination of independently movable emitter and detector arms mounted on a C-arm.

The C-arm 30 is arranged around a structure to be imaged (for example a rotor blade) 20 such that the upper arm 40 is above the upper surface 120 of the structure 20, and the lower arm 50 is below the structure.

The C-arm 30 is supported off the ground by a support means 110, which is approximately indicated as a scissor lift. The support means 110 may take a different form such as a hydraulic ram, or other such devices where the height of the C-arm is adjustable.

The support means 110 is located on a trolley 80 which includes a base 90 and four wheels 100 for movement thereof.

The support means may be arranged to lift and/or lower the C-arm 30 above the trolley 80 so as to place it around the structure 20.

A controller 60 is arranged on the trolley connected to the C-arm by cable 50, although the controller may be arranged in the C-arm or remote from the apparatus 10.

The upper arm 40 includes one or more X-ray emitters, and the lower arm 50 includes a digital X-ray detector. It is contemplated that these positions can be reversed if desirable. In use, the X-ray emitter is controlled by the controller 60 to emit X-rays through the structure 20 such that they are detected by the detector in the lower arm 50.

In use, the trolley 80 is movable along the length of the structure 20 such that the entire structure 20 may be imaged in this way.

The C-arm 30 may be sized such that the entire width of the structure 20 fits under the upper arm 40 and above the lower arm 50. In this regard, the term “width” may mean the dimension lying in the plane parallel to the ground surface and perpendicular to the longitudinal length of the structure 20.

Alternatively, if the width of the structure is too great to completely fit under the upper arm, the apparatus 10 may be relocated relative to the structure 20, such that the open end of the C-arm is on the opposite side of the structure from that shown in FIG. 1. However, it is to be appreciated that other arrangements of the emitters and detector panel are envisaged such that they may be movable independently, or otherwise not limited by the structure of a C-arm.

Wheels 100 are arranged on opposite sides of the trolley such that the trolley 80 is movable along the longitudinal length of the structure 20. However, it is contemplated that other wheels, or movement means may be included instead of, or as well as, the wheels 100 shown. In this way, the trolley 80 may be more easily positionable. For instance, continuous tracks may be provided. The wheels may be motorised such that, in use, the trolley 10 moves along under the longitudinal length of the structure 20. The wheels could be replaced with continuous tracks. The wheels/tracks may be steerable such that the apparatus 10 is not only moveable along the longitudinal length of the structure 20 but is also positionable width-wise of the structure.

The apparatus 110 may be controlled such that its speed of movement relative to the structure 20 matches that of its image capturing.

Also, the support means 110 may be rotatable, about a vertical axis, relative to the trolley deck 90 so that the open end of the C-arm 30 may be positioned, as required, relative to the structure 20. The apparatus 10 is configured such that no force is applied to the structure 20 during imaging, for instance, by the apparatus 10 touching the structure 20.

The apparatus 10 may be controlled such that its speed of movement relative to the structure 20 matches that of its image capturing. The apparatus may be configured to stop at each X-ray image acquisition site.

The apparatus 10 is arranged to move in all directions within a horizontal plane parallel to the ground surface.

1. A method of producing a tomogram, the method comprising the steps of:
acquiring a plurality of first x-ray attenuation images of an object over a first region;
reconstructing a first three-dimensional density function indicative of attenuation of x-ray radiation from the plurality of first x-ray attenuation images;
identifying a first plurality of fiducial markers in the first three-dimensional density function;
acquiring a plurality of second x-ray attenuation images of the object over a second region;
reconstructing a second three-dimensional density function indicative of attenuation of x-ray radiation from the plurality of second x-ray attenuation images;
identifying a second plurality of fiducial markers in the second three-dimensional density function;
comparing relative locations of the first plurality of fiducial markers with relative locations of the second plurality of fiducial markers, and thereby identifying a plurality of common fiducial markers present in the first plurality of fiducial markers and the second plurality of fiducial markers;
using locations of the plurality of common fiducial markers in the plurality of first x-ray attenuation images and the plurality of second x-ray attenuation images to determine an offset between the first region and the second region; and
reconstructing a combined three-dimensional density function indicative of attenuation of x-ray radiation from the plurality of first x-ray attenuation images and the plurality of second x-ray attenuation images.

2. The method of producing a tomogram of any claim 1, wherein reconstructing the combined three-dimensional density function comprises determining an emitter offset and/or a detector offset between first position(s) from which the plurality of first x-ray attenuation images is acquired and second position(s) from which the plurality of second x-ray attenuation images is acquired.

3. The method of producing a tomogram of claim 1, further comprising identifying a combined plurality of fiducial markers in the combined three-dimensional density function.

4. The method of producing a tomogram of claim 1, further comprising allocating as a combined plurality of fiducial markers, the first plurality of fiducial markers and the second plurality of fiducial markers.

5. The method of producing a tomogram of claim 3, further comprising the steps of:
acquiring a third set of x-ray attenuation images of the object over a third region;
identifying a third plurality of fiducial markers in the third set of x-ray attenuation images;
comparing relative locations of the combined plurality of fiducial markers with relative locations of the third plurality of fiducial markers, and thereby identifying a further plurality of common fiducial markers present in the combined plurality of fiducial markers and the third plurality of fiducial markers;
using locations of the further plurality of common fiducial markers to determine a further offset between the first and/or second region and the third region; and
reconstructing a further combined three-dimensional density function indicative of attenuation of x-ray radiation from the plurality of first x-ray attenuation images, the plurality of second x-ray attenuation images and the third set of x-ray attenuation images.

6. The method of producing a tomogram of claim 5, wherein the third set of x-ray attenuation images comprises a single third x-ray attenuation image only.

7. The method of producing a tomogram of claim 5, wherein the third set of x-ray attenuation images comprises a plurality of third x-ray attenuation images.

8. The method of producing a tomogram of claim 7, further comprising the step of reconstructing a third three-dimensional density function indicative of attenuation of x-ray radiation from the third set of x-ray attenuation images, and wherein the step of identifying the third plurality of fiducial markers in the third set of x-ray attenuation images comprises identifying the third plurality of fiducial markers in the third three-dimensional density function.

9. The method of producing a tomogram of claim 5, wherein reconstructing the further combined three-dimensional density function comprises determining a further emitter offset and/or a further detector offset between first and/or second position(s) from which the plurality of first and/or plurality of second x-ray attenuation images are acquired and third position(s) from which the third set of x-ray attenuation images is acquired.

10. A system for producing a tomogram, the system comprising:
an x-ray detector panel;
an x-ray emitter panel comprising an array of x-ray emitters permanently fixed with respect to each other, each x-ray emitter configured to emit a respective cone of x-rays therefrom to impinge on the x-ray detector panel; and
a processor configured to operate the x-ray detector and emitter panels to carry out the method of claim 1.
